(19) United States
(12) Patent Application Publication
(10) Pub. No.: US 2018/0035762 A1
(43) Pub. Date: Feb. 8, 2018

(54) MEASURING AN OBJECT

(71) Applicant: C & J CLARK INTERNATIONAL LIMITED, Street, Somerset (GB)

(72) Inventors: Chris TOWNS, Taunton, Somerset (GB); Peter RICKETT, Windsor, Berkshire (GB); Samuel JONES, Windsor, Berkshire (GB)

(21) Appl. No.: 15/549,779

(22) PCT Filed: Feb. 8, 2016
(86) PCT No.: PCT/EP2016/052596

§ 371 Date: Aug. 9, 2017

(30) Foreign Application Priority Data
Feb. 9, 2015 (GB) 1502119.9
May 15, 2015 (GB) 1508414.8

(51) Int. Cl. A43D 1/02; A61B 5/00; A61B 5/107; G01B 11/02; G06T 7/00

(52) U.S. Cl. CPC A43D 1/025; G01B 11/02; G06T 7/001; A61B 5/1074; A61B 5/1079; A61B 5/6898; G06T 2207/30196

(57) ABSTRACT

A measurement apparatus comprising a base for receiving an object to be measured is disclosed. The base is provided with a pattern that is visually different in different locations. An abutment element is moveable relative to the object and the pattern, the moveable abutment element being configured to partially cover the pattern from an imaging apparatus and provide at least one datum point for the imaging apparatus for determining measurement data for the object based on the at least one datum point and a part of the pattern remaining visible for the imaging apparatus. Methods, imaging apparatuses and a system for generating and processing measurement data are also disclosed.

The present invention relates to measuring an object, for example a foot or hand. The measurement can be provided particularly but not exclusively for the purpose of determining the correct size of footwear or another piece of clothing.

Measuring devices such as a footgauge have been used for measuring feet. Conventionally a footgauge comprises a graduated scale which is read e.g. by a staff member in shoe shop after a foot has been placed between two opposing abutments. However, users might wish to have a more convenient and yet accurate way of determining the size of the foot.

There may also be a desire to conveniently convert the measurement readings into data that can be readily communicated and/or stored in a remote location such as in a server. The possibility of communicating and/or collecting measurement data can be found useful for example by users of internet shopping and/or shoe shops and manufactures who may wish to have a better knowledge of their customer base and potential customers.

In addition to foot measurements, similar issues can apply to other fields where easy to take and accurate measurements and conversion of the measurement results to data would be desired. Thus these issues are not limited to measurement of a particular object such as a foot.

Embodiments of the invention aim to address one or several of the issues related to measurement of objects by a measurement device.

According to an aspect there is provided a measurement apparatus comprising a base for receiving an object to be measured, a pattern on the base, the pattern being visually different in different locations, and an abutment element moveable relative to the object and the pattern, wherein the moveable abutment element is configured to partially cover the pattern from an imaging apparatus and provide at least one datum point for the imaging apparatus for determining measurement data for the object based on the at least one datum point and a part of the pattern remaining visible for the imaging apparatus.

According to an aspect there is provided a handheld device comprising data processing apparatus and a camera, the device being configured to determine measurement data for an object based on at least one image of a measurement apparatus, wherein the device is configured to capture at least one image of at least one datum point provided by a moveable abutment and a visible part of a pattern on the base of the measurement apparatus, and determine measurement data for the object based on the at least one image of the at least one datum point and the visible part of the pattern.

According to an aspect there is provided a method of measuring an object by a measurement apparatus comprising a base, a pattern associated with the base, the pattern being visually different in different locations, an imaging device, and an abutment element moveable relative to the base and providing at least one datum point for the imaging device, wherein, in use, the abutment element is placed in a predefined relation with an object to be measured such that the abutment element partially covers the pattern while a part of the pattern remains visible for the imaging device, the method comprising capturing at least one image by the imaging device of the at least one datum point and the visible part of the pattern, and determining measurement data for the object based on the at least one image of the at least one datum point and the visible part of the pattern.

According to an aspect there is provided an application for a computer comprising code means adapted to perform, when the program is run on processor apparatus, a method comprising receiving image data from a camera pointed towards an abutment element of a measurement apparatus as described herein, and processing the image data comprising at least one captured image of at least one datum point provided by an abutment element and a visible part of a pattern on the measurement apparatus, the pattern being partially covered by the abutment element to determine measurement data for the object based on the at least one image.

In accordance with a more specific aspect the at least one datum point comprises at least one edge of an opening provided in the abutment element. The at least one datum point may also comprise at least one edge of a recess provided in the abutment element and/or at least one edge of a wall of the abutment element and/or at least one side edge of an element protruding from the abutment element.

A pattern on the base can comprise a randomised array of subpatterns and/or a picture.

A second pattern may be provided on the abutment element in the vicinity of the at least one datum point. This pattern can be used for focusing the imaging apparatus. The second pattern may comprise a logo.

At least one magnet may be provided for holding the abutment element and the base in a predefined relation.

A base and an abutment element can provide a cooperative teething structure for alignment of the abutment element in at least one direction. The teething structure can comprise thin grooves and ribs or projections on at least one of the co-operative surfaces. The pattern can be produced on the teething on the base.

The measurement apparatus can comprise a footgauge.

The handheld device can comprise a touch screen device and/or a mobile phone. The handheld device can comprise an application adapted to determine size of a foot and/or a shoe based on the at least one image.

A system comprising at least one measurement apparatus as described herein, at least one imaging apparatus comprising a communication interface and a camera, and a remote data processing apparatus configured for processing and/or storing data received from the at least one imaging apparatus may also be provided. Information from an imaging device determined based on the at least one image can be communicated to the remote data processing apparatus. The remote data processing apparatus can be configured to analyse data received from a plurality of imaging apparatus over the time to determine changes over the time, trends or location dependent variations in measured objects.

In some arrangements the pattern is provided on a flat base, a first abutment projecting upwardly from the level of the base and a carriage being slidable along the flat base. Conveniently, the carriage provides an opening which overlies the pattern and through which, in use, a camera can view a part of the pattern from above. In certain constructions a support portion for holding the camera is disposed above the pattern.

The footgauge can be flat-packed and foldable into an operative condition.

Various exemplifying embodiments of the invention are described below with reference to the attached drawings. Steps and elements explained herein may be reordered, omitted, and combined to form different embodiments and any step indicated as performed may be caused to be performed by another device or module. In the drawings:

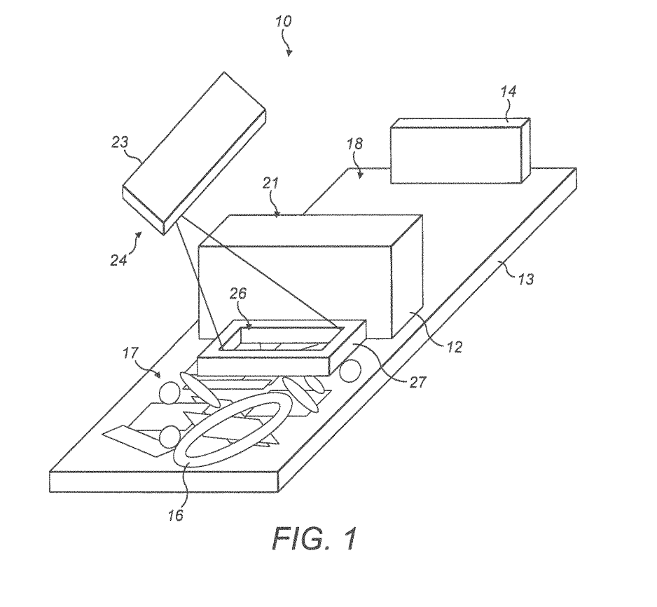

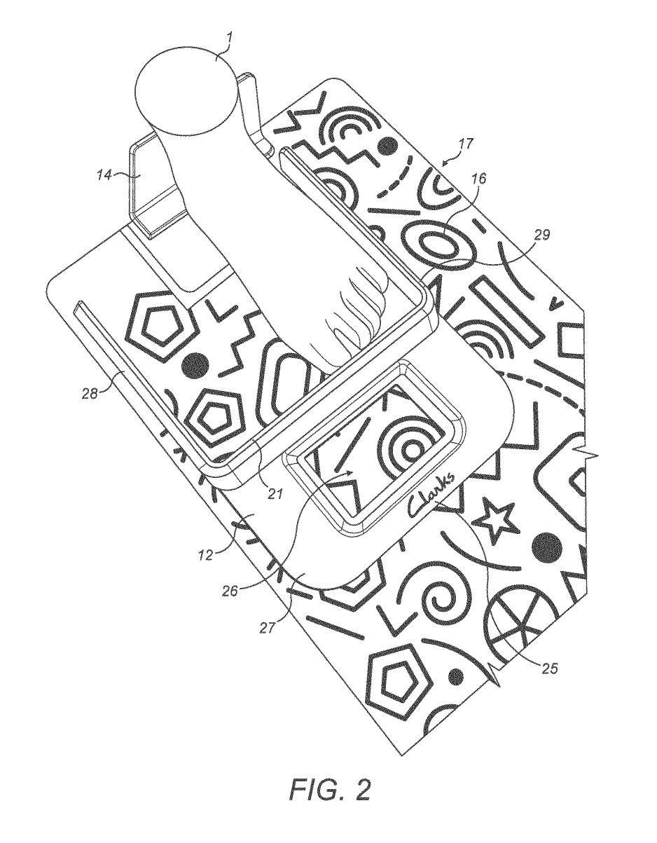

FIGS. 1 and 2 show examples of measurement apparatus in accordance with herein disclosed principles,

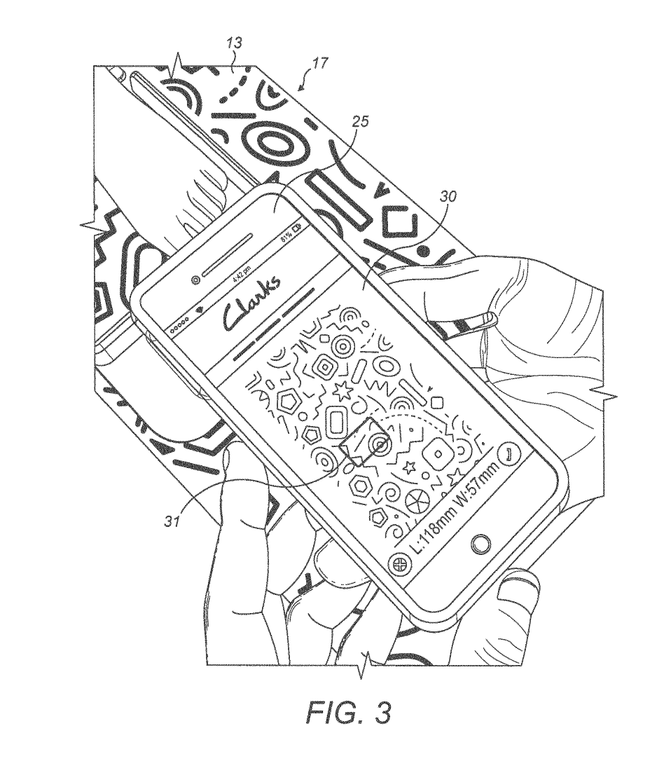

FIG. 3 shows an example of a display on an imaging apparatus,

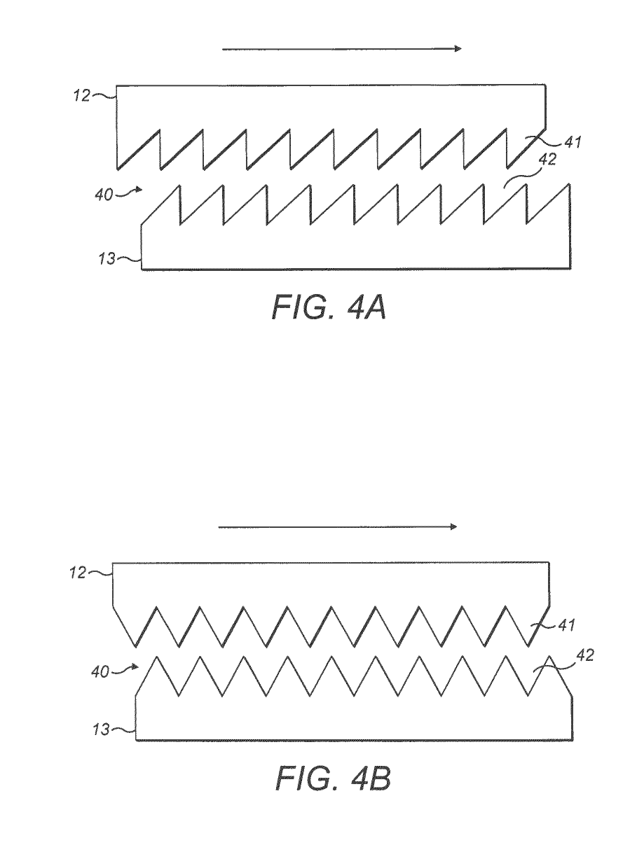

FIGS. 4 to 6 show examples of alignment and/or holding structures,

FIG. 7 is a flowchart in accordance with one form of operation,

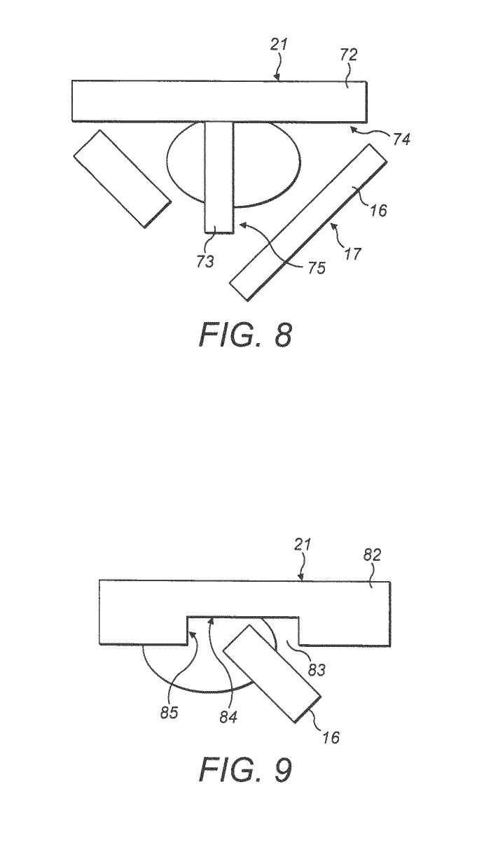

FIGS. 8-10 show further examples of abutment elements, and

FIG. 11 shows an example of control apparatus for determining measurements.

In the following certain detailed examples of a measurement apparatuses and methods are described with reference to the appended Figures. Some of the examples are described with reference to measurement of a foot but it is noted that similar principles can be applied to measurement of other objects.

FIG. 1 illustrates an example of a measurement device 10 comprising a base-plate 13 and an end wall 14 of the base arrangement. The end wall provides a first abutment 14 of the measurement device 10. A moveable abutment element or carriage 12 is also provided. The back wall of the carriage 12 provides a second abutment 21 which faces the first abutment 14. Walls or abutments 14 and 21 define there between a measurement space 18.

The carriage 12 has a generally flat base to enable the carriage 12 to sit on and slide along the base-plate 13 generally towards or away from the abutment 14 of the base arrangement. In certain embodiments the carriage can also move sideways. An example of this will be explained later with reference to FIG. 2.

A measurement pattern 17 is provided for use in measurements of at least the distance between the first and second abutments 14, 21. The measurement pattern is arranged on the base plate such that it is visually different in different locations. This can be provided by means of an irregular pattern of differently shaped and/or sized objects, or subpatterns. In this example the measurement pattern 17 comprises a random array of different shapes and smaller patterns 16. The pattern can be provided by various techniques on the surface of the base plate. For example, a pattern can be produced on the base during manufacture of e.g. a plastic base plate. Printing, painting, placing a sticker are other non-limiting examples of producing the pattern on a base.

The carriage 12 comprises an imaging opening or window 26. The window opens towards the base plate so that a part of the pattern 17 is visible through the window 26 while other parts are covered by the carriage. The pattern can be any appropriate pattern which enables determination of the position of the abutment 21 of the carriage 12 relative to the abutment 14. That is, the pattern is such that unique location dependent views can be provided through the window in different locations of the carriage on the base plate. The difference can be provided based regular shapes placed irregularly on the base and/or irregular shapes, symbols, markings, colours, characters and so forth. FIGS. 1 and 2 show examples of irregular patterns consisting of different shapes.

The arrangement is such that at least one image of the pattern can be captured through the window 26 by an imaging device 23. The imaging device can comprise an appropriate device provided with a camera (not visible in FIG. 1) and data processing device for processing image data. Imaging by a camera pointed to the window is depicted by camera view 24. Thus the camera can capture through the window arranged to move relative to the pattern a unique location dependent view of a part of the pattern. Based on the captured image at least one measurement of an object placed in the measurement space 18 can then be determined in a predetermined manner.

The imaging device 23 can advantageously be a handheld device. In certain examples a Touchscreen Computer Device (TCD) 23 is used. TCD can be in the form of a mobile phone, or “smart phone”, having a built-in camera 24 which is pointed to the opening. Although a touchscreen device can be advantageously used, a more basic mobile phone having a camera can also be used although some functionality as described later may be lost. A device comprising the camera can also be a larger device than a mobile phone, such as a tablet computer device. A digital camera with data processing capabilities may also be used. It is therefore noted that although in a particular example a reference is made to a touch screen device (TCD) with a camera, the camera can be included in any appropriate device.

The measurement device 10 can comprise a footgauge for measuring feet, particularly for determining the correct size of footwear for humans. It is common for feet to be measured in store using one of a variety of foot measuring devices but increasingly shoes are being sold via the internet where the step of foot measurement in store may not be an option. However, it is of great importance for footwear to fit correctly so as to be comfortable and to avoid injuries and pain caused by poorly fitting footwear. In accordance with an embodiment a foot placed in the measurement space 18 is measured based on at least one image taken of the window 26. Shoe size indication can then be determined based on the at least one image. Determining the at least one measurement can be based on information of the pattern and the relation of the part visible through the windows to the edges of the window. The edges can be used as the datum point of the window. For example, the device can calculate via a suitable application (“App”; a computer program loaded on to the device) the required shoe size based on the position of the window 26 relative to the pattern taking into account the known distance of the window 26 from the second end abutment 21.

The determining is based on differently patterned and/or coloured areas of the measurement pattern 17 on the base plate 13. The opening, or rather at least one edge thereof provides at least one datum point that is used to determine the location of the abutment element relative to the base.

Processing of information based on the datum point and view obtained by imaging through the window can be provided at the device comprising the camera. Alternatively, a part of the processing can be provided in a remote location such as a server. Also, least a part of the processed information may be communicated from the device comprising the camera for use elsewhere. For example, data can be communicated to a remote server.

FIG. 1 shows a use scenario where a user holds a user device 23, for example a smart phone or a tablet, in hand and points it towards the window 26. Although the image may be somewhat blurry, the image processing software can determine from the distinguishable patterns visible in the window the position of the window, and hence the carriage, and the image by the handheld device can be sufficient, depending on the application.

FIG. 2 illustrates an example where a carriage 12 is used to measure both the width and length of a foot 1 placed on a patterned base plate 13. In this example, the heel of the foot 1 is placed against the back wall 14. One of the side walls 28, 29 of the carriage 12 is placed against one side of the foot. The end wall or abutment 21 of the carriage is placed against the toes. At this stage a first image of the window 26 is captured. The carriage is then moved sideways so that the other side wall touches the foot. At this stage a second image is captured.

FIG. 3 shows a display 30 on a touchscreen device 23 when the carriage is on the second position, i.e. position of FIG. 2. The pointer box 31 indicated the location on the window on the pattern. The length and width of the object, in this example a foot, as computed by an application running on the device based on the two images is shown at the bottom of the display. The shoe size can then be determined based on these dimensions.

A handheld device can apparently be held at different distances and angles relative to the window, and focusing and/or alignment of the camera may be desired. An arrangement for focusing a handheld camera can be as follows. The carriage 12 can be provided with a distinctive pattern 25 that is placed close to the window 26. In the example of FIG. 2 such as pattern is provided by the stylised Clarks® logo. The application running on the device 23 is aware of the exact dimension and shape of the logo, and therefore can determine its position relative to the window and adjust its the operation accordingly. Thus, when the camera is pointed to the window the application can lock to the logo or other distinctive focusing pattern and therefore also to the window.

Use of the logo as the focusing pattern has the advantage that no additional patterns need to be provided and the appearance of the device can be kept clean. However, it is noted that this is only an advantageous example and that there are various other ways of providing a focusing pattern. For example, the edges of the window can be provided with appropriate focusing markings.

Part 27 of the carriage providing the window 26 can be relatively thin. In addition of making the design light, this enables wider angles for the picture taking. Also, in applications where the camera is supported on the carriage the support for the camera device can arranged on part 27, even if it is relatively thin. Although a camera put on top of a thin support part, and thus relatively close to the pattern, may produce a blurred image, this can give accurate enough information for the processing apparatus to determine information about the size of the object to be measured.

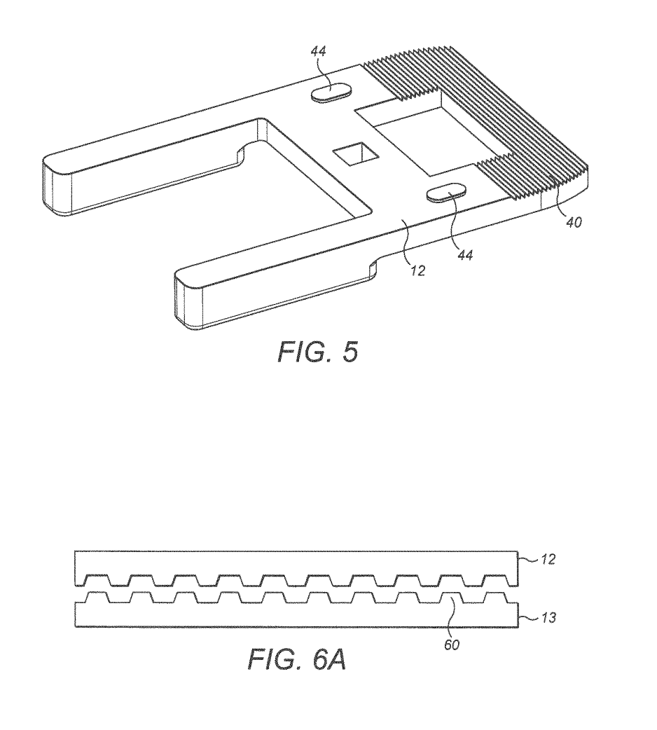

In accordance with an aspect an alignment arrangement for ensuring linear movement and proper alignment of the carriage 12 on the flat base plate 13 is provided. This can be provided by an appropriate co-operating teething. FIGS. 4A and 4B shows sectioned views of a part of arrangements comprising thin ribs and grooves that mesh with each other. FIG. 5 shows schematically a rib and groove formation arranged on a part of the bottom surface of the carriage 12. In this arrangement the top surface of the base plate 13 and the bottom surface of the carriage 12 are provided with cooperating formations 40 comprising thin cooperative ribs 41 and grooves 42. In FIG. 4A a saw tooth type arrangement is shown whereas FIG. 4B shows a symmetrical rib and tooth arrangement.

FIGS. 4A and B also indicate by an arrow the direction of movement towards the object to be measured. It is noted that the drawings are illustrative only and show the ribs and grooves to be larger in relation to the base plate and the carriage than what these might be in an advantageous implementation. To illustrate, in accordance with a non-limiting example the ribs can be in the range of about 0.5 to 1.5 mm high. The size of the teething can dependent on the desired increments of the measurements, and can vary from this example.

In FIG. 5 the grooves and ribs are shown to extend sideways. Such formations can also extend in the longitudinal direction.

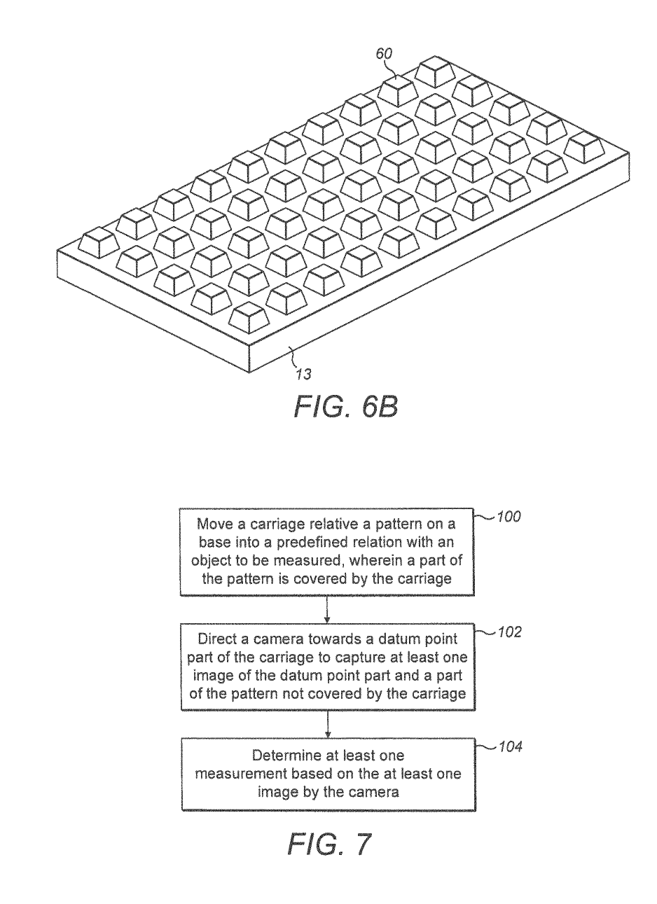

Variations of the principle of having a teethed alignment structure are also possible. FIGS. 6A and 6B show an example where, instead of cooperating grooves and ribs, movement preventing teething is provided on the base 13 and carriage 12 by projections 60. FIG. 6A shows a cross-sectional view of such arrangement and FIG. 6B shows an isometric view of the base plate 13. This arrangement allows movement and locking in two directions.

A possibility is to arrange the teething between the sliding surfaces by discontinuous ridges and small round or conical projections that co-operate such that the carriage can be moved in both directions, i.e. along the grooves and at places through the gaps in the ridges.

The formations can be e.g. moulded or otherwise formed in the cooperating surfaces during manufacture of the components. This enables easy and costs effective manufacture of the alignment arrangement. Another advantage of the thin rib formations is that a pattern with continuous appearance can be produced on top of the surface during moulding or otherwise. The top surface will appear smooth and clean as reed for rails, deep grooves or the like alignment structures can be avoided.

In accordance with a possibility a magnetic field can be provided between the carriage 12 and the base plate 13 to hold them together. To provide this at least one magnet may be provided in the carriage and/or the base plate. At least one magnetic or metallic strip extending in the direction of movement of the carriage may be provided in the base for holding and guiding and aligning a carriage sliding thereon. Use of at least two magnets and cooperating strips can be used for the alignment of the carriage. FIG. 5 shows an example of magnets 44 provided in the carriage 12. The magnets draw the carriage towards a metallic layer provided in the base plate. Magnet(s) can be positioned ion a variety of manners in the base plate and/or the carriage. The magnet(s) and metallic counterparts can be visible or encased in the material.

Other types of alignment and holding arrangements may also be provided. For example, a base plate may also be provided with rails and the carriage may be provided with co-operative counterparts that engage with the rails, or the base plate can have grooves and the carriage can have protruding elements with slot into the grooves.

FIG. 7 shows a flowchart for operation in accordance with an example for measuring an object by a measurement apparatus as described herein. In the method an abutment element such as a carnage comprising an end wall is moved at 100 relative to the pattern on the base into a predefined relation with the object to be measured. At this position a part of the pattern is covered by the carriage, as shown e.g. by FIG. 2. At least one image is the captured at 102 by the imaging device directed to the at least one datum point and the visible part of the pattern. Measurement data for the object can then be determined at 104 based on the at least one image of the at least one datum point and the visible part of the pattern.

The imaging device can be focused and/or aligned prior to capturing of an image by pointing the imaging device to a second pattern provided on a surface of the abutment element in the vicinity of the at least one datum point.

Thin cooperative grooves and ribs extending along the top of the base and bottom of the abutment element can be used for aligning the abutment element on the base.

Other arrangements for providing at least one datum point and for indicating the location of the moveable carriage relative to the pattern than an opening provided by means of a window are also possible. FIGS. 8-10 show a few examples. In FIG. 8 an abutment element 72 providing an end abutment 21 comprises a protruding element 73 extending from a first datum point surface 74 and providing a second datum point surface 75. Surfaces 74 and 75 can extend substantially normal to each other. This provides an X,Y coordinate system enabling determination of the location of the carriage 72 based on elements 16 of the pattern and their relative location to surfaces 74 and 75. If needed, one or more of the surfaces on the other side of the protruding element 73 can also be used in the measurement.

In FIG. 9 an abutment element 82 is shown where the protruding element of FIG. 8 is replaced by a recess 83. The recess provides surfaces 84 and 85 that can be used to determine the location of the abutment element 82 based on imaging of the recess and features 16 of the pattern 17 that is partially covered by the abutment element and partially visible in the recess.

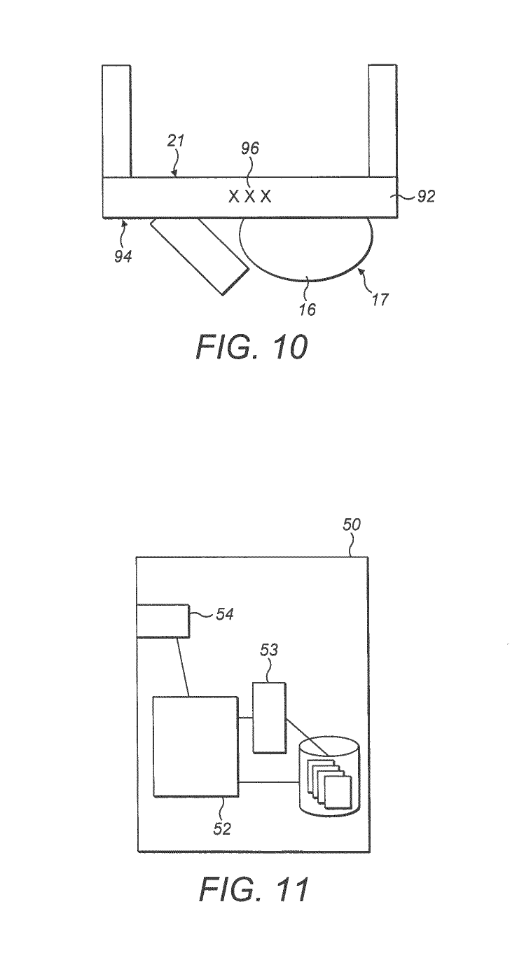

FIG. 10 shows yet another possible arrangement where only one surface 94 of an abutment element 92 is configured for use in determining a measurement of an object based on image data by an imaging apparatus. As above, the abutment element covers a part of the features 16 of the pattern 17 while other parts remain visible. One edge surface can be sufficient in certain applications, in particular if only the distance between the end abutment 21 and an opposing end abutment needs to be determined.

According to a possibility a further pattern 96, for example a logo, is provided on the carriage 92. This further pattern can be used to provide a sideways reference point and/or a focusing pattern for the imaging apparatus.

FIG. 11 shows an example of control apparatus for a device capable of processing image data from a camera and for determining at least one measurement of a foot or another object based on images of the pattern and scale. The control apparatus 50 can be for example integrated with, coupled to and/or otherwise controlling the camera and/or device such as a touchscreen computer or a mobile phone comprising the camera. For this purpose the control apparatus comprises at least one memory 51, at least one data processing unit 52, 53 and an input/output interface 54. Via the interface the control apparatus can be coupled to the camera and/or a communication apparatus, for example a wireless transceiver. The control apparatus can be configured to execute an appropriate software code to provide the control functions. This includes execution of the applications (“Apps”) discussed earlier. The control apparatus can also be interconnected with other control entities.

According to an aspect data based on images by the imaging apparatus is communicated to a remote data processing apparatus. For example, data can be communicated from a device comprising the camera over an Internet Protocol (IP) based network to a remote server to order wearable items. The items can comprise, for example shoes, a hat, gloves and so forth items where measurement information is needed to ensure that a fitting item is ordered and delivered.

Data of the measurements may also be collected at a remote database for example for use in marketing and/or customer analysis. The data may be associated with a user profile, and used later e.g. for further purchasing transactions. Data collected from a number of measurements over the time can also be used for general analysis. For example, the collected data can be analysed to recognise changes in foot shapes and/or sizes over time. For example, understanding if the average foot of the shoe consuming public has got longer and/or increased in width and girth can be valuable for a manufacturer of shoes. Also, the collected data can be used to recognise national and/or geographical characteristics and/or trends.

The various embodiments and their combinations or subdivisions may be implemented as methods, apparatuses, or computer program products. Methods for downloading computer program code for performing the same may also be provided. Computer program products may be stored on non-transitory computer-readable media, such as memory chips, or memory blocks implemented within the processor, magnetic media such as hard disk or floppy disks, and optical media such as for example DVD and the data variants thereof, CD, magnetic disk, or semiconductor memory. A client application performing the method at a user device comprising a digital camera can be downloaded from an appropriate server. Method steps may be implemented using instructions operable to cause a computer to perform the method steps using a processor and a memory. The instructions may be stored on any computer-readable media, such as memory or non-volatile storage.

The required data processing apparatus may be provided by means of one or more data processors. The described functions at each end may be provided by separate processors or by an integrated processor. The data processors may be of any type suitable to the local technical environment, and may include one or more of general purpose computers, special purpose computers, microprocessors, digital signal processors (DSPs), application specific integrated circuits (ASIC), gate level circuits and processors based on multi core processor architecture, as non-limiting examples. The data processing may be distributed across several data processing modules. A data processor may be provided by means of, for example, at least one chip. The memory or memories may be of any type suitable to the local technical environment and may be implemented using any suitable data storage technology, such as semiconductor based memory devices, magnetic memory devices and systems, optical memory devices and systems, fixed memory and removable memory. In general, the various embodiments may be implemented in hardware or special purpose circuits, software, logic or any combination thereof. Some aspects of the invention may be implemented in hardware, while other aspects may be implemented in firmware or software which may be executed by a controller, microprocessor or other computing device, although the invention is not limited thereto.

While various aspects of the invention may be illustrated and described as block diagrams, flow charts, or using some other schematic pictorial representation, it is well understood that these blocks, apparatus, systems, techniques or methods described herein may be implemented in, as non-limiting examples, hardware, software, firmware, special purpose circuits or logic, general purpose hardware or controller or other computing devices, or some combination thereof.

In the examples of FIGS. 1 and 2 an array of randomly arranged small patterns provided the measurement pattern on the base. In accordance with a possibility the pattern comprises a picture, such as a photograph or a picture of an artistic creation. The measurement determination software can be trained to the recognisable features and dimensions of the picture so that that it can recognise different locations on the picture based on features of the picture particular to that location. This training can even be provided on a per user basis, that is, a user can have a customised pattern on the base. A pattern in the form of a picture can then be used to determine the location of the abutment element based on the location of the datum point and visible parts of the picture. This enables e.g. use of individualised measurement patterns. The pattern may also comprise numbers, or other characters and/or a scale.

In accordance with a possibility a measurement pattern is arranged to be movable relative to a fixed abutment element and a camera is arranged to capture the movement of the pattern relative to the fixed abutment element. For example, a camera supported on a support portion fixed to the body of the measurement device or the fixed abutment element can be pointed towards a moveable pattern. A datum point can be provided in the body and/or the fixed abutment of the device so that the pattern slides relative to the datum point. Such arrangements may be desired e.g. in applications where the movable carriage shall be of simple construction.

According to a possibility the pattern is arranged on a retractable platform, e.g. on a roll arranged in association with the movable or fixed abutment element. One end of the platform is fixed relative to one of the abutments, and thus movement of one of the abutment causes the pattern to roll out when the distance increases and in when the distance decreases. A camera can be directed close to the roll end of the free pattern to capture an image of the pattern visible through an appropriately positioned window or otherwise relative to a fixed datum point.

The measurement device does not need to be placed in use on a surface, for example a floor. Instead, the base can be held against an object to be measured. For example, an infant may not stand still on a footgauge but rather the footgauge needs to be held against his/hers foot. An arrangement to held the foot against the base may be provided to enable capturing of the necessary images.

The base and the moveable abutment element can be manufactured from a variety of materials by a variety of techniques. The base and/or the abutment element can be made from any plastic, aluminium or another metal allow, wood, rubber and so forth.

It will also be appreciated that the measurement devices may be assembled from a flat condition from cardboard and/or other sheet materials. The assembly may require interlocking/interengaging flaps in order to ensure sufficient rigidity or other methods of securement may be utilised such as adhesives, adhesive tapes, sections of hook/loop fastener or other fasteners. E.g. a footgauge may also be incorporated in, and folded from, an adapted shoe box using suitable predetermined fold lines and perforations etc. such that it can be used next time a pair of shoes is required. This is particularly relevant for children's shoes as the child's feet grow with age.

A footgauge may be dimensioned so as to measure each foot individually or as a pair. When measuring a pair the parallel movement of the two abutments will ensure that the foot measurement is made on the longest foot so as to optimise the fit of the footwear. The foot size determination can comprises measurement and determination of the length and/or width and/or girth of a foot.

The foregoing description provides by way of exemplary and non-limiting examples a full and informative description of exemplary embodiments of the invention. However, various modifications and adaptations may become apparent to those skilled in the relevant arts in view of the foregoing description, when read in conjunction with the accompanying drawings and the appended claims. All such and similar modifications of the teachings of this invention will still fall within the spirit and scope of this invention.

1. A measurement apparatus comprising:
a base for receiving an object to be measured,
a pattern on the base, the pattern being visually different in different locations, and
an abutment element moveable relative to the object and the pattern,
wherein the moveable abutment element is configured to partially cover the pattern from an imaging apparatus and provide at least one datum point for the imaging apparatus for determining measurement data for the object based on the at least one datum point and a part of the pattern remaining visible for the imaging apparatus.

2. (canceled)

3. The measurement apparatus as claimed in claim 1, wherein the at least one datum point comprises at least one edge of an opening provided in the abutment element, and/or at least one edge of a recess provided in the abutment element and/or at least one edge of a wall of the abutment element and/or at least one side edge of an element protruding from the abutment element.

4. The measurement apparatus as claimed in claim 1, wherein the pattern comprises a randomised array of subpatterns or a picture.

5. (canceled)

6. The measurement apparatus as claimed in claim 1, further comprising a second pattern provided on the abutment element in the vicinity of the at least one datum point for use in focusing the imaging apparatus.

7. (canceled)

8. The measurement apparatus as claimed in claim 1, wherein the base and the abutment element are configured to provide a cooperative teething structure for alignment of the abutment element.

9. The measurement apparatus as claimed in claim 8, wherein the teething structure comprises thin grooves and ribs or projections on the co-operative surfaces, and the pattern is produced on the teething on the base.

10. (canceled)

11. (canceled)

12. The measurement apparatus as claimed in claim 1, comprising at least one magnet for holding the abutment element and the base in a predefined relation.

13. The measurement apparatus as claimed in claim 1, comprising a footgauge.

14. A handheld device comprising data processing apparatus and a camera, the device being configured to determine measurement data for an object based on at least one image of a measurement apparatus comprising a base for receiving an object to be measured, a pattern on the base, the pattern being visually different in different locations, and an abutment element moveable relative to the object and the pattern and configured to partially cover the pattern and provide at least one datum point for determining measurement data for the object, wherein the handheld device is configured to
capture at least one image of the at least one datum point provided by the moveable abutment and a visible part of the pattern on the base of the measurement apparatus, and
determine measurement data for the object based on the at least one image of the at least one datum point and the visible part of the pattern.

15. The handheld device as claimed in claim 14 comprising a touch screen device and/or a mobile phone.

16. A system comprising:
at least one measurement apparatus, wherein the measurement apparatus comprises a base for receiving an object to be measured, a randomized pattern on the base, the randomized pattern being visually different in different locations, and an abutment element moveable relative to the object and the randomized pattern and configured to partially cover the randomized pattern and provide at least one datum point for determining measurement data for the object,
at least one imaging apparatus comprising a communication interface and a camera, and
a remote data processing apparatus configured for processing and/or storing data received from the at least one imaging apparatus.

17. The system according to claim 16, wherein the remote data processing apparatus is configured to analyse data received from a plurality of imaging apparatus over the time to determine changes over the time, trends or location dependent variations in measured objects.

18. A method of measuring an object by a measurement apparatus comprising a base, a pattern associated with the base, the pattern being visually different in different locations, an imaging device, and an abutment element moveable relative to the base and providing at least one datum point for the imaging device, wherein, in use, the abutment element is placed in a predefined relation with an object to be measured such that the abutment element partially covers the pattern while a part of the pattern remains visible for the imaging device, the method comprising:
capturing at least one image by the imaging device of the at least one datum point and the visible part of the pattern, and
determining measurement data for the object based on the at least one image of the at least one datum point and the visible part of the pattern.

19. The method as claimed in claim 18, wherein the at least one datum point comprises at least one edge of an opening provided in the abutment element, at least one edge of a recess provided in the abutment element, at least one edge of a wall of the abutment element and/or at least one side edge of an element protruding from the abutment element.

20. The method as claimed in claim 18, comprising focusing the imaging device by pointing the imaging device to a second pattern provided on a surface of the abutment element in the vicinity of the at least one datum point.

21. The method as claimed in claim 18, comprising aligning the base and the abutment element by means of a plurality of small cooperative elements along the top of the base and bottom of the abutment element.

22. The method as claimed in claim 18, comprising:
placing a first side wall and an end wall of the abutment element in contact with respective two sides of the object to be measured and subsequently capturing a first image of the at least one datum point and the visible part of the pattern,
moving the abutment element sideways so that a second side wall comes into contact with a second side of the object and subsequently capturing a second image of the at least one datum point and the visible part of the pattern, and
determining the length and width of the object based on the images.

23. The method as claimed in claim 18, comprising communicating information from the imaging device determined based on the at least one image.

24. The method as claimed in claim 23, comprising collecting the information from a plurality of imaging devices over time and analysing the collected information to determine changes over the time, trends or location dependent variations in the measured objects.

25. The method as claimed in claim 18, comprising measuring a foot.

26.-31. (canceled)
